(19) United States
(12) Patent Application Publication
(10) Pub. No.: US 2025/0237647 A1
(43) Pub. Date: Jul. 24, 2025

(54) SELF-ADMINISTERED SYSTEM FOR BREAST CANCER SCREENING AND DIAGNOSIS USING BIOMARKER-SPECIFIC ANALYSIS

(71) Applicants: MADHWA HG RAJ, METAIRIE, LA (US); PRAKASH N. RAO, MCKINNEY, TX (US)

(72) Inventors: MADHWA HG RAJ, METAIRIE, LA (US); PRAKASH N. RAO, MCKINNEY, TX (US)

(21) Appl. No.: 19/033,666

(22) Filed: Jan. 22, 2025

Related U.S. Application Data
(60) Provisional application No. 63/624,079, filed on Jan. 23, 2024.

(51) Int. Cl. G01N 33/543; G01N 33/574; G16H 50/20

(52) U.S. Cl. CPC G01N 33/54388; G01N 33/57415; G01N 33/57423; G01N 33/57442; G01N 33/57449; G16H 50/20

(57) ABSTRACT

The present invention provides a versatile, self-administered system for early detection and monitoring of breast cancer and its subtypes using a lateral flow assay platform. It includes a sample pad for a fingerstick blood sample, a conjugate pad with labelled detection reagents, a test zone with six lines (one control, five biomarker-specific), and an absorbent pad for capillary flow. These biomarkers allow precise subtype identification, enabling early diagnosis and personalized treatment. A smartphone app with AI and machine learning analyses biomarker patterns, offering qualitative and quantitative results. Designed to be user-friendly, minimally invasive, and cost-effective, the system is ideal for at-home or point-of-care use and supports treatment monitoring and detection of other cancers like ovarian and lung.

### FIELD OF INVENTION

The present invention relates to a self-administered system for the early detection and monitoring of breast cancer and its subtypes, utilizing a lateral flow assay platform capable of analyzing clinically relevant biomarkers from a minimally invasive blood sample, such as one obtained via fingerstick. The system is designed for at-home use or in field settings, enabling qualitative and/or quantitative analysis of protein and nucleic acid biomarkers with visual or mobile application-based interpretation, thereby facilitating timely medical intervention and personalized healthcare.

### BACKGROUND OF INVENTION

Breast cancer remains one of the most prevalent and life-threatening forms of cancer worldwide, with a significant impact on morbidity and mortality rates. Current screening techniques, such as mammography, while effective in detecting tumors in early stages, are subject to limitations including the need for specialized equipment, trained professionals, and regular appointments, making these methods inaccessible to many, especially in resource-limited settings. Furthermore, the lack of frequent monitoring increases the likelihood of undetected rapid disease progression, which can result in late-stage diagnoses with reduced survival rates.

At the molecular level, breast cancer is a heterogeneous disease characterized by varying subtypes, each associated with specific biomarkers. Established biomarkers such as CA 125, CA 15-3, CA 27.29, CEA, and HER 2 Neu are clinically relevant for breast cancer diagnosis, as their levels in the blood have been shown to be selectively elevated depending on the disease subtype. However, these markers are not exclusive to breast cancer and can also be elevated in other conditions, such as ovarian or lung cancers, as well as some non-cancerous diseases. This overlap necessitates a screening tool that can accurately detect and interpret biomarker patterns to identify breast cancer specifically and guide the user to seek timely medical advice.

The majority of currently available screening methods for breast cancer detection involve invasive procedures, require large blood sample volumes, and depend on sophisticated laboratory equipment to evaluate relevant biomarkers. For instance, blood tests conducted in clinical settings necessitate skilled personnel for sample collection, processing, and interpretation, leading to delays in diagnosis and increased costs. These challenges make routine screening difficult, particularly in resource-limited or remote areas. Furthermore, such methods often fail to facilitate frequent and early detection, which is critical for improving treatment outcomes.

Mammography, a widely recommended screening tool for breast cancer, presents additional limitations. It relies on ionizing radiation, which poses potential health risks with repeated exposure, and often detects structural changes in breast tissue rather than early molecular or cellular changes. Additionally, mammograms can be uncomfortable for patients, leading to poor compliance with regular screening guidelines.

These challenges underscore the need for a minimally invasive, cost-effective, and user-friendly tool that enables frequent screening and potential diagnosis for breast cancer. Such a solution should allow early detection of molecular changes indicative of cancer while eliminating the discomfort, radiation exposure, and dependence on specialized equipment associated with mammography.

Numerous advancements have been made in lateral flow assay technologies for screening purposes. For example, U.S. Pat. No. 6,436,651 discloses a lateral flow immunoassay device for detecting analytes in a biological fluid using labeled antibodies. While this technology provides a foundation for rapid screening assays, it primarily focuses on single-analyte detection and does not address the simultaneous detection of multiple biomarkers associated with a complex disease like breast cancer. Furthermore, the patent lacks provisions for integrating detection of nucleic acids such as mRNA, which is a critical aspect of the present invention for enhancing screening and diagnostic accuracy and specificity.

Similarly, U.S. Pat. No. 7,858,396 describes a lateral flow test strip featuring numerous capture zones arranged in a linear array perpendicular to the flow of the sample, thereby enabling simultaneous analyses of multiple analytes. However, its focus is primarily on the spatial arrangement of capture zones to facilitate efficient multi-analyte detection. The invention also does not contemplate the use of biomarker panels specific to breast cancer subtypes, nor does it address the challenges of minimizing false positives due to biomarker overlap with other cancers or non-cancerous conditions. Further, the described subject matter is more suited for laboratory or clinical applications, lacking the user-centric design suitable for at-home use without the need for specialized expertise.

Another relevant prior art, Japanese Patent Application No. 2007504842, provides screening kits for assessing the presence, progression, or risk of developing breast cancer, as well as for evaluating the efficacy of therapeutic agents. The described invention focuses on kits comprising reagents such as antibodies, antibody fragments, nucleic acid probes, or a combination thereof, which are designed to bind to specific markers associated with breast cancer. However, the focus remains on providing a detection kit intended for clinical use and involve laboratory-based assessments of biomarker expression levels to diagnose or monitor breast cancer or to test therapeutic efficacy, requiring specialized expertise and equipment. In contrast, the present invention provides a self-administered system specifically designed for qualitative and/or quantitative detection of specific breast cancer biomarkers, including proteins and nucleic acids, using a single drop of blood obtained via a minimally invasive fingerstick at-home or field use, eliminating the need for laboratory equipment or specialized personnel.

There is a need for a system and method for identifying subtypes, prognosis, and monitoring of breast cancer in a simplified manner is critical, as it would make frequent screening and diagnosis practical even in resource-limited settings. To address this to certain extent, the inventors of the US Patent Application No. US20220178924 provides non-invasive methods for determining breast cancer subtypes by isolating extracellular vesicles from a biofluid sample and analysing differential protein or peptide expression therein.

However, this method requires the isolation of extracellular vesicles, a process that typically necessitates specialized laboratory equipment and expertise, making it unsuitable for use outside clinical or laboratory settings. The prior art focuses on subtype determination and monitoring of treatment efficacy or recurrence, relying on complex biomarker panels to provide such information. Furthermore, the methods described in the prior art primarily detect proteins or peptides within extracellular vesicles. While effective for these purposes, the methodology lacks the accessibility and simplicity.

The present invention addresses these limitations by providing a self-administered system based on a lateral flow assay platform. This system utilizes a single drop of blood obtained via a fingerstick and is capable of detecting the presence or absence of one or more biomarkers relevant to breast cancer. The assay is optimized to visually or digitally indicate elevated biomarker levels, enabling early detection and timely medical intervention without requiring elaborate equipment or specialized expertise.

The invention further incorporates advanced biomarker detection technologies, including the use of monoclonal antibodies for protein detection and aptamer-based systems for messenger RNA (mRNA) analysis. This flexibility allows for the simultaneous detection of multiple biomarkers, individually or in combination, in a single assay. Additionally, the system can be coupled with artificial intelligence and machine learning algorithms to interpret biomarker patterns, enabling subtype characterization and personalized therapy recommendations, ensuring accurate and personalized guidance, and empowering users to seek timely medical intervention. By simplifying the screening process and making it accessible for at-home use, the present invention offers a transformative solution for improving breast cancer detection and outcomes. This combination of technical advancements, user-friendliness, and clinical relevance demonstrates the non-obviousness and significant contribution of the present invention over the known prior art.

### OBJECT OF THE INVENTION

It is the principal object of the invention, to provide a self-administered system for the detection and monitoring of breast cancer, utilizing a lateral flow assay platform that is user-friendly, cost-effective, minimally invasive, and suitable for use in non-clinical settings. The invention aims to facilitate early detection by identifying clinically relevant biomarkers in a single drop of blood obtained through a fingerstick procedure.

Another object of the invention is to provide a screening system capable of detecting both protein and nucleic acid biomarkers, such as mRNA, either individually or in combination, for enhanced screening accuracy and specificity. The invention employs optimized reagents, including monoclonal antibodies and aptamers, to provide qualitative and/or quantitative analysis of biomarkers relevant to breast cancer and its subtypes.

It is a further object of the invention to integrate advanced interpretation capabilities into the system for screening and diagnosis, allowing test results to be visually interpreted directly on the device or through a smartphone application with artificial intelligence (AI) and machine learning algorithms, ensuring timely medical guidance, including subtype characterization and personalized therapy recommendations, without the need for specialized laboratory equipment or expertise.

Yet another object of the invention, is to provide a self-administered system configured as a kit, comprising essential components for early detection and monitoring of breast cancer and its subtypes. The kit includes a lateral flow assay device, a sterile lancet, a buffer solution and a bio-safe disposal container.

An additional object of the invention is to minimize the reliance on invasive procedures, large sample volumes, and ionizing radiation, thereby providing a safe, non-invasive, and radiation-free alternative for cancer diagnosis. The invention further ensures that the system is accessible and practical for frequent screening and diagnosis, particularly in resource-limited settings. By eliminating the need for laboratory equipment and specialized expertise, the system promotes widespread deployment and affordability, addressing critical barriers to effective cancer detection and monitoring.

Another object of the invention is to enable patients to monitor the effectiveness of breast cancer treatment by periodically testing biomarker levels using the self-administered system. The intensity or presence of test lines corresponding to specific biomarkers can indicate treatment progress or disease recurrence, empowering patients to participate actively in their healthcare management.

### SUMMARY OF INVENTION

A self-administered system for breast cancer detection and monitoring, designed to improve patient outcomes across various stages of cancer diagnosis and treatment are disclosed. There is a need for such a system that provides an effective, comprehensive solution by enabling precise detection of clinically relevant biomarkers while minimizing dependence on expensive, invasive, or complex screening procedures. The invention not only facilitates early detection of breast cancer but also supports subtype characterization and treatment monitoring, thereby empowering patients with actionable insights into their health. By offering a cost-effective, user-friendly, and minimally invasive alternative to traditional methods, the present invention transforms screening and diagnostic practices and significantly improves patient outcomes in breast cancer care.

According to the main aspect of the invention, there is provided a self-administered system for the early detection and monitoring of breast cancer and other cancers, designed to be simple, cost-effective, and suitable for use at home or in the field, employing a lateral flow assay capable of detecting one or more biomarker, separately or in combination, using a small quantity of biological sample.

According to another aspect of the invention, there is provided a self-administered system comprising six test lines, wherein one test line serves as a control to ensure the validity of the assay, and the remaining five test lines are embedded with reagents specific to one or more biomarkers selected from the group consisting of CA 125, CA 15-3, CA 27.29, CEA, HER 2 Neu, CDK4, BCL2, BAX, mutant T p53, CD44, Survivin, MYLIP, ARC protein, GADD45, and CDKN1C, allowing simultaneous detection and analysis of biomarker levels, aiding in the early screening and diagnosis of breast cancer and providing insights into its subtype for timely and effective medical intervention.

According to yet another aspect of the invention, the selected biomarkers CA 125, CA 15-3, CA 27.29, CEA, and HER 2 Neu, are emphasized for their clinical relevance in breast cancer diagnosis, as their levels in the biological sample have shown to be selectively elevated depending on the breast cancer subtype.

According to yet another aspect of the invention, there is provided a self-administered system integrated with reagents such as monoclonal antibodies for protein detection and labelled aptamers for detecting messenger RNA (mRNA) corresponding to the biomarkers, enhancing the accuracy and specificity of the system, allowing it to identify early changes in biomarker levels indicative of cancer.

According to another aspect of the invention, the screening and diagnostic system optimized to detect multiple biomarkers individually or in combination using a single test zone, wherein elevated biomarker levels are indicated visually by the intensity of the test line, allowing for easy interpretation without requiring a direct comparison with a control line.

According to another aspect of the invention, the system incorporates advanced interpretation capabilities, including digital analysis through a smartphone application or other digital devices, utilizing artificial intelligence (AI) and machine learning algorithms to analyze biomarker expression patterns, enabling accurate subtype characterization and providing personalized medical recommendations.

According to yet another aspect of the invention, the system is specifically designed for accessibility and frequent use, requiring minimal technical expertise. Its cost-effective design and user-friendly operation ensure suitability for resource-limited settings, making the invention practical for widespread adoption.

According to yet another aspect of the invention, there is provided a self-administered system that supports monitoring the effectiveness of breast cancer treatments by observing changes in biomarker levels over time.

According to another aspect of the invention, there is provided a self-administered system configured as a kit, comprising essential components for early detection and monitoring of breast cancer and its subtypes. The kit includes a lateral flow assay device, a sterile lancet, a buffer solution and a bio-safe disposal container.

According to yet another aspect of the invention, the self-administered system is applicable to other cancers, such as ovarian, uterine, and lung cancers, by leveraging the same biomarker panel, making the system versatile and valuable in diagnosing and monitoring a range of cancers beyond breast cancer.

These and other aspects of the present invention are set out in the following detailed description of the invention.

### BRIEF DESCRIPTION OF THE DRAWINGS

FIG. 1: Illustrates a schematic representation of the self-administered system of the present invention, showing the arrangement of the lateral flow assay device being a test strip (100) comprising of sample pad (101), conjugate pad (102), test zone (103), control zone (104) and wicking pad (105).

FIG. 2: Depicts the self-administered system (200) with multiple test zones (202), each corresponding to a specific biomarker, and the control zone (203) for validating the assay performance.

FIG. 3: Illustrates the workflow of the self-administered system, starting from sample collection via fingerstick, application of the sample to the assay, result interpretation and safe disposal.

The drawings described herein above are for illustration purposes only and are not intended to limit the scope of the present disclosure in any way.

### DETAILED DESCRIPTION OF THE INVENTION

The present invention addresses the need for a self-administered system that provides early detection and monitoring of breast cancer and other cancers by utilizing clinically relevant biomarkers. This system leverages a lateral flow assay platform to detect elevated biomarker levels from a small, minimally invasive blood sample obtained via a simple fingerstick. By identifying specific biomarkers associated with breast cancer subtypes, the system facilitates timely medical intervention, improves the accuracy of cancer diagnosis, and reduces reliance on invasive procedures, large sample volumes, and complex laboratory equipment. The invention enhances accessibility and affordability, ensuring that frequent screening can be conducted, particularly in resource-limited settings. Additionally, the system minimizes the need for ionizing radiation, providing a safe, non-invasive alternative for monitoring cancer progression and treatment efficacy, ultimately improving patient outcomes and empowering users to take an active role in their healthcare management. Features and aspects of the invention are disclosed throughout and include those summarized herein.

In the following disclosure, it is to be understood that description will describe the invention in relation to the preferred embodiments of the invention. All embodiments provided are illustrative and the invention is in no way limited to these preferred embodiments as they are purely to exemplify the invention only and non-limiting representatives of many possible embodiments would be readily apparent without departing from the scope of the invention.

In the context of the present invention, the term “biomarker” refers to a biological molecule, including but not limited to proteins, peptides, nucleic acids (e.g., mRNA), metabolites, or other analytes, whose presence, absence, or altered expression level in a biological sample provides an indicator of a biological or pathological state. Biomarkers may be indicative of diseases, such as cancer, their subtypes, progression, or therapeutic response. Specific biomarkers referenced in this invention include CA 125, CA 15-3, CA 27.29, CEA, HER 2 Neu, and others associated with breast cancer diagnosis and subtype characterization.

As used herein, the term “lateral flow assay” refers to a screening platform in which a liquid sample is applied to a test device, typically comprising a porous material, wherein the liquid flows laterally through the material by capillary action. This flow facilitates interactions between the sample components and embedded reagents, such as antibodies or nucleic acid probes, resulting in a detectable signal. The assay may include one or more test lines and a control line to ensure the validity of the results.

As used herein, the term “lateral flow” refers to the movement of a liquid through a material by capillary action in a horizontal direction. However, it should be understood that this term applies to the liquid's flow from the point where it is applied to another lateral position, even if the device is oriented vertically or at an angle.

As used herein, the term “capillary flow” refers to the movement of liquid where all dissolved or dispersed components travel at approximately the same rate and with minimal disruption, flowing laterally through the membrane without requiring external forces such as pressure or vacuum.

As used herein, the term “test line” refers to a designated region on the screening device where specific reagents, such as monoclonal antibodies or aptamers, are embedded to interact with target biomarkers in the sample. The interaction produces a detectable signal, such as a visible line, indicating the presence or absence of one or more biomarkers associated with breast cancer or other conditions.

As used herein, the term “control line” refers to a region on the screening device embedded with reagents that validate the proper functioning of the assay. The appearance of the control line confirms the successful flow of the sample and reagent interactions, ensuring the reliability of the test results. In the context of the present invention, the control line is configured to ensure assay reliability by validating the proper flow of the sample and the functionality of the reagents. The control line can incorporate various elements aligned with the invention's goals, including but not limited to the following: (1) binding reagents for universal interaction, such as anti-species antibodies or secondary antibodies, which bind to labelled antibodies or detection antibodies used in the assay, ensuring proper migration through the strip and confirming their functionality; (2) a specific biotin-streptavidin interaction, wherein streptavidin is embedded in the control line and biotin-labelled reagents on the conjugate pad confirm successful migration and reaction; (3) Protein A or Protein G coating, which binds to the Fc region of antibodies, irrespective of specificity, thereby validating the presence and movement of functional antibodies through the assay; (4) synthetic polymers or nanoparticles with universal binding properties that interact with conjugate pad labelled particles, ensuring the flow and reagent reactivity; (5) an enzyme-substrate reaction, wherein a pre-loaded enzyme substrate in the control line interacts with enzyme-labelled reagents from the conjugate pad to provide a visible signal, indicating enzymatic activity and migration; (6) a dual-labelled detection system that detects dual labels, such as fluorescent and colorimetric markers, to ensure redundancy in validating the assay's reliability; (7) an alignment verification zone that serves as a positional marker, verifying uniform capillary flow, proper test line readings, and migration; and (8) a calibration standard comprising known concentrations of reagents or standardized particles to generate a fixed signal intensity and validate consistency across multiple tests. These configurations ensure proper sample flow, reagent interaction, user confidence, and diagnostic reliability, thereby enabling reliable and accurate at-home diagnostic capabilities for breast cancer screening and subtype identification.

As used herein, the term “biological sample” refers to a sample derived from a biological source, including but not limited to blood, serum, plasma, saliva, or other body fluids. In the context of the present invention, the sample is typically a small volume of blood, such as one obtained via a minimally invasive fingerstick, used to detect biomarkers associated with breast cancer.

As used herein, the term “minimally invasive” refers to procedures or methods that require minimal intrusion into the body to obtain a biological sample. In the context of this invention, it specifically refers to obtaining a blood sample via a simple fingerstick, avoiding the need for venipuncture or other invasive techniques.

As used herein, and in the appended claims, the term “artificial intelligence” (AI) refers to computational systems and algorithms designed to analyze data patterns, such as biomarker expression levels, to provide diagnostic insights, subtype characterization, and personalized therapy recommendations. AI algorithms are incorporated in the diagnostic system to enhance the interpretation of test results and improve accuracy.

As used herein, and in the appended claims, the term “subtype characterization” refers to the process of identifying specific subtypes of breast cancer based on the expression patterns of selected biomarkers. Subtype characterization aids in tailoring therapeutic strategies and predicting disease prognosis.

As used herein, the term “self-administered” refers to a system or process designed to be operated independently by an individual without the need for specialized training, professional supervision, or external assistance. In the context of the present invention, “self-administered” specifically relates to the ability of a user to collect a biological sample, such as a drop of blood obtained via fingerstick, apply the sample to the diagnostic device, and interpret the results visually or with the aid of a smartphone application. The design, components, and operation of the system are optimized to ensure ease of use, safety, and accuracy, thereby enabling effective cancer screening and diagnosis in non-clinical settings such as the home or remote environments.

Unless defined otherwise, all technical and scientific terms used herein have the same meanings as commonly understood by a person of ordinary skill in the relevant art of the present disclosure. Further, all components listed by generic name, if any, are herein meant to include or encompass all equivalents for such components available.

As used in the specification, and in the appended claims, the singular forms “a”, “an”, “the”, include plural referents unless the context clearly dictates otherwise. Further, the terms such as “may,” “may provide for,” and “it is contemplated that the present invention may” and so forth are terms used in interchange with the terms “is,” “can,” “will,” and like terms used as synonyms.

It is to be understood that the reference, such as “in one embodiment” or “an embodiment” in the disclosure is intended to indicate that at least an embodiment of the invention is included with a particular feature or characteristic described in connection with the embodiment. In various places in the disclosure, it is not necessary that the presence of the phrase “in one embodiment” or “an embodiment” is all referring to the same embodiment.

Further, it is to be understood that the term “comprise” and variations thereof, such as “comprising”, “comprises” and “comprised” as used in this disclosure, except where the context requires otherwise, are not intended to exclude other additives, components, integers or steps.

According to an embodiment of the present invention, a self-administered system for early detection and monitoring of breast cancer and its subtypes is provided to address the limitations of current screening and diagnostic methods, including invasive procedures, large sample volume requirements, and the reliance on sophisticated laboratory equipment.

Further, in an embodiment of the present invention, the system utilizes a lateral flow assay platform to analyze clinically relevant biomarker to detect breast cancer and its subtypes from a small blood sample, such as one obtained via fingerstick, enabling timely medical intervention and improved healthcare outcomes.

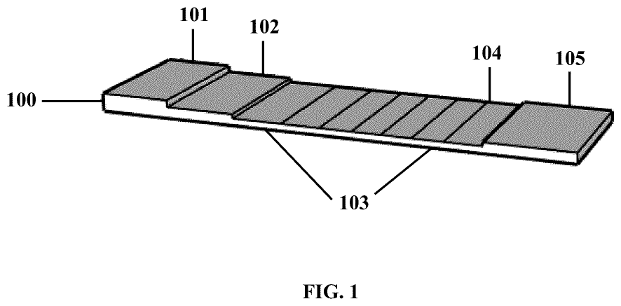

Further, in an embodiment of the present invention, the system utilizing the lateral flow assay platform includes a sample pad for the application of the blood sample, a conjugate pad pre-loaded with labeled detection reagents, a test zone containing the test and control lines, and an absorbent pad to facilitate continuous flow. The arrangement of these components is schematically illustrated in FIG. 1. The assay leverages capillary action to transport the liquid sample through the test strip without the need for external pressure or vacuum.

Further, in an embodiment of the present invention, the system utilizing lateral flow assay platform comprises six test lines, where one test line serves as a control to validate the assay's performance by ensuring that the sample has successfully flowed through the device and the reagents have reacted appropriately. Such control line acts as an internal validation mechanism to confirm the accuracy and reliability of the assay results, thereby enhancing diagnostic reliability. The remaining five test lines are embedded with reagents specific to one or more biomarkers selected from the group consisting of CA 125, CA 15-3, CA 27.29, CEA, HER 2 Neu, CDK4, BCL2, BAX, mutant T p53, CD44, Survivin, MYLIP, ARC protein, GADD45, and CDKN1C. This comprehensive selection of biomarkers ensures that the system is capable of detecting a wide range of clinically relevant molecular changes, enabling a robust screening and diagnostic capability for breast cancer and its subtypes.

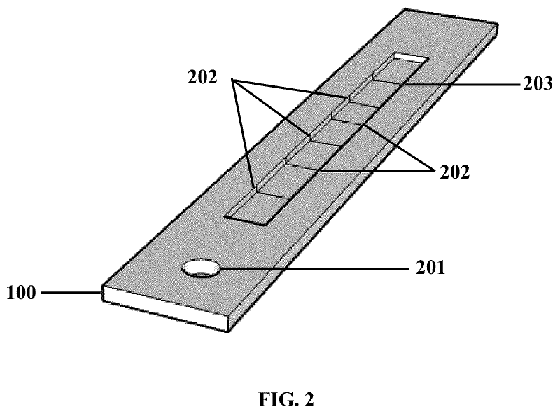

Further, in an embodiment of the present invention, the system is specifically configured to prioritize clinically validated biomarkers associated with breast cancer subtypes, where the five test lines embedded with reagents in the lateral flow assay platform are tailored to detect biomarkers, including but not limited to CA 125, CA 15-3, CA 27.29, CEA, and HER 2 Neu, which have shown to exhibit selective elevation in blood samples depending on the specific breast cancer subtype, thereby enabling precise identification and subtype characterization while enhancing diagnostic specificity and ensuring actionable insights for early detection and personalized medical intervention. The placement of multiple test lines corresponding to specific biomarkers is illustrated in FIG. 2.

Further, in an embodiment of the present invention, the system employs optimized reagents, including monoclonal antibodies for protein detection and labeled aptamers for detecting messenger RNA (mRNA) corresponding to the biomarkers, tailored to enhance specificity and stability, ensuring accurate detection of biomarkers even at low concentrations. Compared to conventional reagents, the optimized reagents minimize cross-reactivity and provide robust performance across diverse environmental conditions, significantly improving the reliability of the screening and diagnostic outcomes. This dual detection capability enhances the specificity and sensitivity of the assay, allowing for the identification of early molecular changes indicative of cancer progression.

The test lines on the system are strategically positioned within the test pad to ensure optimal interaction with the sample as it migrates through the lateral flow assay device. Each test line is specifically configured to detect one or more biomarkers associated with breast cancer subtypes, facilitating targeted and accurate diagnosis. The reagents specific to these biomarkers, including monoclonal antibodies and labeled aptamers, are embedded on the conjugate pad. This arrangement allows for the immediate and efficient binding of the biomarkers present in the sample, enhancing the sensitivity and specificity of the assay. The precise placement of test lines and the embedding of optimized reagents on the conjugate pad ensure that the system delivers reliable and reproducible results. This design is integral to the present invention as it enables a user-friendly, minimally invasive, and highly accurate platform for careening and diagnosis, making it suitable for at-home use and resource-limited settings.

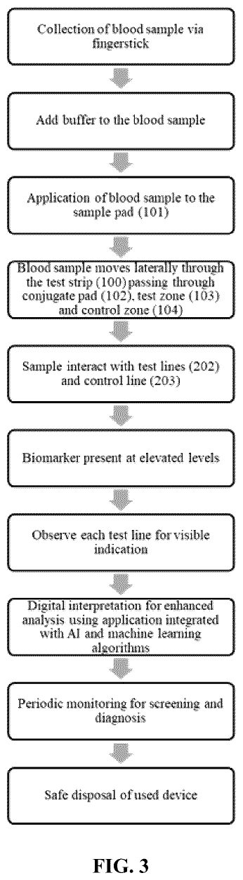

In another embodiment of the present invention, the process for screening and diagnosing breast cancer and its subtypes using the self-administered system of the present invention is provided, which involves several sequential steps designed to ensure accurate, minimally invasive, and user-friendly operation. The workflow of the self-administered system, from sample collection to result interpretation, is illustrated in FIG. 3.
Step 1: Collection of Blood Sample—The process begins with the collection of a small blood sample, typically obtained via a fingerstick. The user applies a drop of blood (approximately 10 to 50 microliters) onto the designated sample pad of the lateral flow assay device. This sample collection method is minimally invasive, eliminating the need for specialized equipment or trained personnel. A drop of a buffer is added to the biological sample, which is configured to ensure adequate sample volume and optimize reaction conditions, including maintaining an appropriate pH level, thereby enhancing the stability and efficiency of the biomarker-detection interactions.
Step 2: Migration of Sample through the Assay Device—Once applied to the sample pad, the blood sample migrates laterally through the assay device via capillary action. The sample first passes through the conjugate pad, which is pre-loaded with labeled detection reagents, such as monoclonal antibodies or aptamers, specific to the biomarkers of interest. The labeled reagents bind to the target biomarkers present in the sample, forming detectable complexes.
Step 3: Interaction with Test Lines and Detection of Biomarkers—As the sample continues to migrate, it reaches the test pad, which contains six test lines. One of these lines serves as a control to validate the assay's performance, while the remaining five lines are embedded with reagents specific to clinically relevant biomarkers, including CA 125, CA 15-3, CA 27.29, CEA, and HER 2 Neu. Each test line interacts with the corresponding biomarker-detection complex, generating a visible result in the form of a line if the biomarker is present at elevated levels.
Step 4: Visualization of Results—The test lines produce visual indications of biomarker levels. Elevated biomarker levels are reflected by the intensity or presence of specific test lines, while the control line confirms the proper functioning of the assay. This visual output provides the user with an immediate qualitative assessment of biomarker presence.
Step 5: Digital Interpretation and Analysis—The system optionally includes a smartphone application integrated with artificial intelligence (AI) and machine learning algorithms. The user captures an image of the assay using their smartphone, and the application analyzes the test and control lines. The AI-powered analysis provides quantitative results, identifies biomarker patterns, and offers diagnostic insights, including subtype characterization and recommendations for medical intervention.

Further, in an embodiment of the present invention, the self-administered system enables simultaneous detection of multiple biomarkers, either individually or in combination, using the same lateral flow assay. The system is capable of detecting both protein biomarkers and nucleic acid biomarkers, such as messenger RNA (mRNA), ensuring a comprehensive diagnostic approach. The dual detection capability enhances the specificity and sensitivity of the assay, making it effective for early-stage cancer detection.

Further, in an embodiment of the present invention, the system is further configured to monitor changes in biomarker levels over time, enabling users to track the effectiveness of breast cancer treatments or detect disease recurrence. Each test line's intensity corresponds to the biomarker level, allowing patients and healthcare providers to observe trends and make informed decisions regarding therapy adjustments.

Further, in an embodiment of the present invention, the biomarker panel in the system is versatile and can be applied to diagnose and monitor other cancers, such as ovarian, uterine, and lung cancers. The biomarkers embedded in the test lines are clinically relevant to multiple cancer types, extending the system's utility beyond breast cancer.
Step 6: Interpretation of Subtype and Recommendations—Based on the biomarker expression patterns identified through the test lines or digital analysis, the system provides a comprehensive report. This report includes the potential subtype of breast cancer, enabling personalized therapy recommendations and facilitating early and targeted intervention.
Step 7: Safe Disposal of Used Device—After the diagnostic process is complete, the system is designed for safe and hygienic disposal. The used device can be sealed in a bio-safe container, minimizing contamination risks and ensuring compliance with safety standards for at-home use.

The process outlined herein emphasizes minimal invasiveness, cost-effectiveness, and ease of use, making it particularly suitable for frequent screening in resource-limited settings. The combination of advanced biomarker detection, AI-driven interpretation, and user-friendly design ensures accurate, timely, and actionable results, addressing critical gaps in current cancer diagnostics.

In another embodiment of the present invention, a self-administered system configured as a kit is provided, comprising essential components for early detection and monitoring of breast cancer and its subtypes. The kit includes a lateral flow assay device, a sterile lancet, a buffer solution and a bio-safe disposal container.

Further, in an embodiment of the present invention, the lateral flow assay device (100) in a kit, consists a sample pad (101) for receiving a blood sample, a conjugate pad (102) pre-loaded with labelled detection reagents specific to biomarkers, a test zone (103) with six test lines—one serving as a control line (203) and the remaining five test lines (202) configured with reagents specific to biomarkers, including but not limited to CA 125, CA 15-3, CA 27.29, CEA, and HER 2 Neu, and an absorbent pad (105) to facilitate continuous capillary flow.

Further, in an embodiment of the present invention, a sterile lancet in a kit is for fingerstick blood collection, a buffer solution is to enhance sample migration and biomarker interaction, and a bio-safe disposal container for hygienic disposal of used components, ensuring compliance with safety standards for at-home or point-of-care use.

The sterile lancet is a single-use device designed to ensure hygienic and safe operation, minimizing the risk of infection or cross-contamination. The lancet features a user-friendly design, including a protective cap and a retractable needle mechanism to prevent accidental injury both before and after use. It complies with regulatory standards for medical devices and provides precise and controlled penetration to obtain an optimal blood sample volume suitable for the system. The sterile lancet is pre-packaged as part of the diagnostic kit and is intended for safe and easy disposal in the accompanying bio-safe disposal container after use. Its inclusion ensures user safety, convenience, and seamless integration into the functionality of the self-administered system.

Further, the system including a buffer solution designed to optimize the reaction conditions required for the lateral flow assay. The buffer solution provides adequate sample volume and maintains the ideal pH and ionic strength to facilitate effective binding and detection of biomarkers. It may be pre-formulated with stabilizing agents, surfactants, or antimicrobial preservatives to enhance the stability of assay components and prevent contamination. The buffer solution is provided in a single-use dropper or vial, ensuring precise and controlled application to the sample pad. The addition of the buffer solution promotes uniform sample flow across the assay device, enhancing both sensitivity and specificity of biomarker detection. Its compact and user-friendly design ensures compatibility with at-home and field-use settings, offering an efficient and reliable diagnostic process.

Furthermore, the bio-safe disposal container referred here may encompass various configurations, including but not limited to, Sealable plastic pouches with self-adhesive closures, rigid containers with tamper-proof lids for secure containment, puncture-resistant boxes for materials with sharp edges or components, or biodegradable bags treated with antimicrobial agents for eco-friendly disposal, which is designed to securely contain materials such as used lateral flow assay strips, biological samples, or any biohazardous waste generated during the diagnostic process, preventing contamination or environmental exposure. The inclusion of a bio-safe disposal container as part of the self-administered system kit enhances the overall safety and usability of the invention, providing users with a comprehensive solution that addresses both diagnostic accuracy and waste management in a seamless manner.

Further, in an embodiment of the present invention, the kit includes a smartphone-compatible application integrated with artificial intelligence (AI) and machine learning algorithms to enhance the interpretation of results obtained from the self-administered system. The kit is configured to provide a comprehensive and user-friendly solution for breast cancer screening and diagnosis, facilitating its use in diverse settings, including but not limited to resource-limited environments.

Further, in an embodiment of the present invention, the system allows for the visual interpretation of results directly on the test strip or through a smartphone application. Elevated levels of specific biomarkers are indicated by the intensity or presence of test lines, while the control line ensures assay validity. The smartphone application, integrated with artificial intelligence (AI) and machine learning algorithms, provides additional interpretation capabilities by leveraging pattern recognition and predictive analytics. These methodologies enable the application to analyze biomarker expression profiles with high accuracy, offering detailed diagnostic insights, including subtype characterization and personalized medical recommendations. This approach ensures robustness in interpretation, even in diverse user environments, by minimizing the potential for error and enhancing the reliability of diagnostic results.

Further, in an embodiment of the present invention, the self-administered system is optimized for frequent use, making it suitable for monitoring treatment progress and disease recurrence. By observing changes in biomarker levels over time, patients can track the effectiveness of their therapy and make informed decisions regarding their healthcare.

The system's versatility extends to its applicability in diagnosing and monitoring other cancers, such as ovarian, uterine, and lung cancers. This adaptability is achieved by leveraging the same biomarker panel used for breast cancer detection, without requiring additional biomarkers. The selected biomarkers demonstrate broad diagnostic relevance, making the system effective for identifying multiple cancer types while maintaining its simplicity and user-friendliness. This is achieved by leveraging the same biomarker panel, demonstrating the invention's broader clinical relevance and utility.

One significant advantage of the present invention is its ability to operate without the need for specialized equipment or trained personnel. The minimal sample volume requirement, coupled with the system's user-friendly design, ensures accessibility in resource-limited settings and promotes widespread adoption.

Another aspect of the invention is its ability to detect multiple biomarkers simultaneously. This multiplexing capability reduces the time and cost associated with separate assays for individual biomarkers, making the system highly efficient and practical for routine screening and diagnosis.

The incorporation of AI and machine learning algorithms further distinguishes the invention from existing technologies. These algorithms analyze biomarker expression patterns to provide comprehensive diagnostic insights, enabling early detection, subtype characterization, and personalized treatment recommendations. The ability of the system to integrate digital analysis ensures accuracy and consistency in result interpretation.

To ensure reliability and robustness, the lateral flow assay components are manufactured using high-quality materials resistant to environmental variations. The reagents are optimized for stability and specificity, ensuring consistent performance under diverse conditions.

The present invention also emphasizes safety and user comfort by eliminating the need for invasive procedures and ionizing radiation. The fingerstick method for sample collection is minimally invasive, reducing discomfort and encouraging regular use for early detection and monitoring.

The invention is particularly advantageous for patients in remote or underserved areas, where access to traditional diagnostic methods is limited. By providing a portable and cost-effective solution, the system addresses critical barriers to effective cancer screening and diagnosis.

The lateral flow assay's capability to detect both protein and nucleic acid biomarkers in a single assay enhances its clinical utility. This dual detection system ensures comprehensive analysis, improving the accuracy and reliability of the results.

The biomarkers embedded in the test lines are selected based on their clinical relevance to breast cancer subtypes. For example, CA 125 and CA 27.29 are associated with specific subtypes, while HER 2 Neu is indicative of aggressive forms of breast cancer. This targeted approach ensures precise and actionable diagnostic information.

The control line serves as an internal validation mechanism, confirming the integrity of the assay. Its presence ensures that the sample has successfully flowed through the test strip and that the reagents have interacted correctly.

The smartphone application accompanying the system provides a user-friendly interface for result interpretation. By capturing an image of the completed assay, the application analyzes the test and control lines to provide qualitative or quantitative results. The use of AI ensures that the analysis is accurate and consistent, regardless of user expertise.

The invention's cost-effectiveness makes it ideal for large-scale deployment in population screening programs. By reducing the dependency on expensive laboratory infrastructure, the system democratizes access to cancer screening and diagnosis.

In summary, the present invention provides a self-administered system for breast cancer screening and diagnosis that combines advanced biomarker detection technologies with user-friendly features. Its ability to deliver accurate, timely, and personalized diagnostic insights ensures significant improvements in cancer detection and patient outcomes. The system's innovative design, coupled with its accessibility and affordability, addresses critical challenges in current diagnostic practices and sets a new standard for cancer care.

The foregoing has been a description of certain non-limiting preferred embodiments of the invention. Those of ordinary skill in the art will appreciate that various changes and modifications to this description may be made without departing from the spirit or scope of the present invention, as defined in the following claims.

We claim:

1. A self-administered system for breast cancer screening and diagnosis, comprising, a lateral flow assay device (100) having:
(a) a sample pad (101) configured to receive a blood sample obtained via fingerstick;
(b) a conjugate pad (102) pre-loaded with labeled detection reagents specific to biomarkers associated with breast cancer;
(c) a test zone (103) comprising six test lines, wherein one test line serves as a control line (203) to validate the assay's performance and the remaining five test lines (202) are embedded with reagents specific to selected biomarkers; and
(d) an absorbent pad (105) configured to facilitate continuous capillary flow of the sample;
wherein the lateral flow assay device is configured to visually indicate elevated levels of biomarkers through the intensity or presence of the test lines.

2. The system as claimed in claim 1, further comprising a smartphone application integrated with artificial intelligence (AI) and machine learning algorithms for analyzing biomarker expression patterns and providing diagnostic insights, including breast cancer subtype characterization.

3. The system as claimed in claim 1, wherein the biomarkers selected for the test lines are selected from the group consisting of CA 125, CA 15-3, CA 27.29, CEA, HER 2 Neu, CDK4, BCL2, BAX, mutant T p53, CD44, Survivin, MYLIP, ARC protein, GADD45, and CDKN1C.

4. The system as claimed in claim 1, wherein the biomarkers selected for the test lines, including but not limited to CA 125, CA 15-3, CA 27.29, CEA, and HER 2 Neu, are specific to at least one subtype of breast cancer, providing subtype-specific diagnostic insights.

5. The system as claimed in claim 1, wherein the biomarkers selected for the test lines exhibits selective elevation in blood samples depending on the specific breast cancer subtype.

6. The system as claimed in claim 1, wherein the test lines are spatially arranged to ensure distinct and non-overlapping visual results for each biomarker.

7. The system as claimed in claim 1, wherein the control line is configured to confirm proper flow of the sample and validate reagent interaction to ensure assay reliability.

8. The system as claimed in claim 1, wherein the labelled detection reagents comprise monoclonal antibodies specific to protein biomarkers and labelled aptamers specific to messenger RNA (mRNA) biomarkers, ensuring dual detection capability.

9. The system as claimed in claim 1, wherein the labelled detection reagents on the conjugate pad are optimized to minimize cross-reactivity and enhance specificity.

10. The system as claimed in claim 1, wherein the smartphone application provides quantitative analysis of biomarker levels by capturing and analyzing an image of the completed assay.

11. A method for self-administered breast cancer screening and diagnosis, comprising the steps of:
(a) collecting a blood sample via a minimally invasive fingerstick;
(b) adding a buffer solution to the collected blood sample;
(c) applying the blood sample to a sample pad of a lateral flow assay device;
(d) allowing the sample to migrate through the device via capillary action, where the sample interacts with labelled detection reagents on a conjugate pad and reacts with test lines embedded with reagents specific to biomarkers;
(e) visually observing the test and control lines for qualitative results; and
(f) optionally analyzing the assay using a smartphone application integrated with artificial intelligence and machine learning algorithms to provide diagnostic insights and subtype characterization.

12. The method as claimed in claim 11, further comprising the step of monitoring changes in biomarker levels over time to track treatment efficacy and detect disease recurrence.

13. The method as claimed in claim 11, wherein the collected sample having a volume of between 10 microliters and 50 microliters.

14. The system as claimed in claim 1, wherein the diagnostic and monitoring capabilities of the assay are further applicable to detecting biomarkers associated with ovarian, uterine, and lung cancers, thereby extending the versatility of the assay for the diagnosis and monitoring of multiple types of cancer.

15. A self-administered diagnostic kit for breast cancer screening and diagnosis, comprising: a lateral flow assay device (100) including a sample pad (101) configured to receive a blood sample obtained via fingerstick, a conjugate pad (102) pre-loaded with labelled detection reagents specific to biomarkers associated with breast cancer, a test zone (103) comprising six test lines, wherein one test line (203) serves as a control to validate assay performance, and the remaining five test lines (202) are embedded with reagents specific to selected biomarkers and an absorbent pad (105) configured to facilitate continuous capillary flow of the sample; a sterile lancet for collecting a blood sample; a buffer solution to enhance sample migration and biomarker interaction; a smartphone-compatible application integrated with artificial intelligence (AI) and machine learning algorithms for analyzing biomarker expression patterns and providing diagnostic insights; and a bio-safe disposal container for hygienic disposal of used components, ensuring safety and ease of use.
